(19) United States
(12) Patent Application Publication
(10) Pub. No.: US 2025/0122129 A1
(43) Pub. Date: Apr. 17, 2025

(54) METHOD OF PREPARING ADJUSTABLE ORGANIC FERTILIZER

(71) Applicant: INDUSTRIAL TECHNOLOGY RESEARCH INSTITUTE, Hsinchu (TW)

(72) Inventors: Hsin-Ju HSIEH, Hsinchu City (TW); Laurensia IRMAYANI, Hsinchu City (TW); Jia-Jun TEE, Tainan City (TW); Shing-Der CHEN, Hsinchu City (TW); Ren-Yang HORNG, Zhubei City (TW); Jer-Young CHEN, Hsinchu City (TW); Wen-Pin HUNG, Miaoli County (TW)

(73) Assignee: INDUSTRIAL TECHNOLOGY RESEARCH INSTITUTE, Hsinchu (TW)

(21) Appl. No.: 18/916,077

(22) Filed: Oct. 15, 2024

Related U.S. Application Data
(60) Provisional application No. 63/590,523, filed on Oct. 16, 2023.

(30) Foreign Application Priority Data
Jun. 6, 2024 (TW) 113121031

(51) Int. Cl. C05F 17/20; C05F 17/50

(52) U.S. Cl. CPC C05F 17/20; C05F 17/50

(57) ABSTRACT

A method for preparing an adjustable organic fertilizer is provided. The method includes providing a ratio of nitrogen, phosphorus, and potassium required for a target crop and collecting a plurality of wastes. The method includes categorizing the fertilizer concentrations of the same type of feeding substrates within the wastes separately, and adjusting the composition of the categorized wastes to form a composite feeding substrate according to the ratio of nitrogen, phosphorus, and potassium required for the target crop. The method includes subjecting the composite feeding substrate to an anaerobic fermentation process. The anaerobic fermentation process includes introducing the composite feeding substrate into an acidification tank to obtain a first mixture and introducing the first mixture into a methanogenesis tank to obtain a second mixture. The method further includes separating the second mixture to obtain liquid digestate and solid digestate, wherein the solid digestate serves as an organic fertilizer.

### CROSS REFERENCE TO RELATED APPLICATIONS

This application claims priority of Taiwan Patent Application No. 113121031, filed Jun. 6, 2024, and the benefit of U.S. Provisional Application No. 63/590,523, filed Oct. 16, 2023, the entirety of which are incorporated by reference herein.

### TECHNICAL FIELD

The present invention relates to a method of preparing an adjustable organic fertilizer.

### BACKGROUND

As the circular economy becomes the mainstream, the issue of how to convert waste into resources and reduce carbon emissions has gradually attracted attention from industry and government. Organic wastes include poultry and livestock manure, waste diatomaceous earth, food processing sludge, brewing sludge, agricultural sludge, animal residues and plant residues, etc. Common organic wastes include livestock waste, agricultural waste, food waste, and so on. Under the environmental protection demands for a sustainable environment, how to effectively handle and utilize organic waste has become an important issue in the current industry.

Taking poultry and livestock manure as an example, depending on the types of poultry and livestock raised, the nitrogen, phosphorus anhydride and potassium oxide contained in the feed will be excreted with the feces. Therefore, poultry and livestock manure contains the nitrogen phosphorus, potassium, other nutrients and organic matter required for general crop growth, so it can be used as a raw material for organic fertilizers. Specifically, nitrogen fertilizer is the main component of producing chlorophyll and can be used as leaf fertilizer. Seedlings and foliage plants have high demand for nitrogen fertilizer. Phosphate fertilizer can promote flower bud differentiation and blooming and can be used as flower fertilizer or fruit fertilizer. Flowering and fruit plants have high demand for phosphate fertilizers. Potassium fertilizer can strengthen the rhizomes and can be used as stem fertilizer or fruit fertilizer. Flower seedlings or adult plants have high demand for potassium fertilizer.

Organic waste has the potential to be used as a raw material for organic fertilizer. If it can be effectively recycled, it can not only reduce the impact on the environment, but also increase soil productivity. However, general organic waste only forms low-concentration solid digestate fertilizer of a single-composition after anaerobic fermentation, which cannot be adjusted according to the fertilization needs of different crops. The content of the solid digestate fertilizer needs to be adjusted through aerobic composting and adding animal and plant meal before it can be applied to the soil for irrigation.

Based on the above, existing methods of utilizing organic waste to prepare organic fertilizer have not yet met the needs in all respects. The development of organic fertilizer preparation methods that can further improve the reuse rate and applicability of organic waste is still an issue of concern in related fields.

### SUMMARY

In accordance with some embodiments of the present disclosure, a method for preparing an adjustable organic fertilizer is provided. The method includes the following steps. The method includes providing a ratio of nitrogen, phosphorus, and potassium required for a target crop and collecting a plurality of wastes. The method includes categorizing the fertilizer concentrations of the same type of feeding substrates within the plurality of wastes separately, and adjusting the composition of the categorized wastes to form a composite feeding substrate according to the ratio of nitrogen, phosphorus, and potassium required for the target crop. The method includes subjecting the composite feeding substrate to an anaerobic fermentation process. The anaerobic fermentation process includes introducing the composite feeding substrate into an acidification tank to obtain a first mixture. The anaerobic fermentation process includes introducing the first mixture into a methanogenesis tank to obtain a second mixture. The acidification tank includes acidifying bacteria. The pH value of the acidification tank is between 3 and 7.5, and the temperature of the acidification tank is between 30° C. and 60° C. The methanogenesis tank includes methanogens. The pH value of the methanogenesis tank is between 6 and 8, and the temperature of the methanogenesis tank is between 30° C. and 60° C. Moreover, the method further includes separating the second mixture to obtain liquid digestate and solid digestate, wherein the solid digestate serves as an organic fertilizer.

In order to make the features or advantages of the present disclosure clear and easy to understand, a detailed description is given in the following embodiments with reference to the accompanying drawings.

### BRIEF DESCRIPTION OF THE DRAWINGS

The present invention can be more fully understood by reading the subsequent detailed description and examples with references made to the accompanying drawings, wherein:

FIG. 1 shows a step flow chart of a method of preparing an adjustable organic fertilizer in accordance with some embodiments of the present disclosure;

FIG. 2A shows a schematic diagram of the temperature changes of the acidification tank and the methanogenesis tank during the anaerobic fermentation process in accordance with an embodiment of the present disclosure;

FIG. 2B shows a schematic diagram of the pH value changes of the acidification tank and the methanogenesis tank during the anaerobic fermentation process in accordance with an embodiment of the present disclosure;

FIG. 2C shows a schematic diagram of the concentration changes of volatile suspended solids (VSS) of the acidification tank and the methanogenesis tank during the anaerobic fermentation process in accordance with an embodiment of the present disclosure;

FIG. 3A and FIG. 3B respectively show the growth conditions of seeds in the control group and the solid digestate sample group in accordance with an embodiment of the present disclosure;

FIG. 3C shows the germination state of seeds in the control group and the solid digestate sample group in accordance with an embodiment of the present disclosure;

FIG. 3D shows the analysis results of the germination index of the control group and the solid digestate sample group in accordance with an embodiment of the present disclosure;

FIG. 4A and FIG. 4B show the results of the pot experiments of lettuce; the pots on the left are the control group cultivated with water; the pots in the middle are the Comparative Example group cultivated with commercially available organic fertilizer (Fwusow Green Fertilizer (4-7-2) of Fwusow Industry Brand, a mixed organic fertilizer, fertilizer item (number): mixed organic fertilizer (5-12)); and the pots on the right are the Example group cultivated with the solid digestate of Example 4;

FIG. 5A and FIG. 5B show the results of the pot experiments of green onion; the pots on the left are the control group cultivated with water; the pots in the middle are the Comparative Example group cultivated with commercially available organic fertilizer (Fwusow Green Fertilizer (4-7-2) of Fwusow Industry Brand, a mixed organic fertilizer, fertilizer item (number): mixed organic fertilizer (5-12)); and the pots on the right are the Example group cultivated with the solid digestate of Example 4;

FIG. 6A shows a schematic diagram of the temperature changes of the acidification tank and the methanogenesis tank during the anaerobic fermentation process in accordance with an embodiment of the present disclosure;

FIG. 6B shows a schematic diagram of the pH value changes of the acidification tank and the methanogenesis tank during the anaerobic fermentation process in accordance with an embodiment of the present disclosure;

FIG. 6C shows a schematic diagram of the concentration changes of chemical oxygen demand (COD) during the anaerobic fermentation process in accordance with an embodiment of the present disclosure;

FIG. 7A shows a schematic diagram of changes in methane production during the anaerobic fermentation process in accordance with an embodiment of the present disclosure;

FIG. 7B shows a schematic diagram of the concentration changes of methane and carbon dioxide during the anaerobic fermentation process in accordance with an embodiment of the present disclosure;

FIG. 8A and FIG. 8B respectively show the growth conditions of seeds in the control group and the solid digestate sample group in accordance with an embodiment of the present disclosure;

FIG. 9A shows the results of the pot experiments of bok choy in an Example;

FIG. 9B shows the results of the pot experiments of bok choy in a Comparative Example;

FIG. 9C shows the results of the pot experiments of bok choy in a Comparative Example;

FIG. 10 shows the growth conditions of seeds in the solid digestate sample group in accordance with an embodiment of the present disclosure;

FIG. 11 shows the results of the pot experiments of small-fruited tomatoes, in which from left to right are the control group, Comparative Example group 1, Comparative Example group 2 and Example group; the potted plants in the control group were cultivated with only water without adding fertilizer; the potted plants in the Comparative Example group 1 were cultivated with a commercially available chemical fertilizer group (nitrophosphorus-based Heiwangte No. 43 organic composite fertilizer of Known-You Brand, with the ratio of nitrogen to phosphorus anhydride to potassium oxide to magnesium oxide to calcium oxide to organic matter being 15:15:15:3:7.5:3); the potted plants in the Comparative Example group 2 were cultivated with a commercially available organic matter fertilizer group (Fwusow green Fertilizer of Fwusow Industry Brand, a mixed organic fertilizer with the ratio of nitrogen to phosphorus anhydride to potassium oxide being 4:7:2); and the potted plants in the Example Group are cultivated with the solid digestate corresponding to small-fruited tomatoes (with the ratio of nitrogen to phosphorus anhydride to potassium oxide being 3:2:2);

FIGS. 12A to 12D respectively show the growth results of outdoor potted tomatoes on the 21st day after planting.

### DETAILED DESCRIPTION

The method of preparing an adjustable organic fertilizer of the present disclosure is described in detail in the following description. It should be understood that in the following detailed description, for purposes of explanation, numerous specific details and embodiments are set forth in order to provide a thorough understanding of the present disclosure. The specific elements and configurations described in the following detailed description are set forth in order to clearly describe the present disclosure. It will be apparent that the exemplary embodiments set forth herein are used merely for the purpose of illustration and not the limitation of the present disclosure.

Unless defined otherwise, all technical and scientific terms used herein have the same meaning as commonly understood by one of ordinary skill in the art to which this disclosure belongs. It should be appreciated that, in each case, the term, which is defined in a commonly used dictionary, should be interpreted as having a meaning that conforms to the relative skills of the present disclosure and the background or the context of the present disclosure, and should not be interpreted in an idealized or overly formal manner unless so defined.

In view of the demand for organic waste reduction faced by the current industry and the market demand for related technical products and services, embodiments of the present disclosure provide a method of using organic waste to prepare adjustable organic fertilizer. The method can adjust the feed composition of organic waste according to the growth needs of the target crops (for example, the required ratio of nitrogen, phosphorus, and potassium) to form a composite feeding substrate with appropriate fertilizer content, thereby optimizing the anaerobic fermentation reaction, and reducing high concentrations of ammonia nitrogen to inhibit the metabolic pathways of anaerobic organisms. Accordingly, the decomposition and stabilization of organic matter can be accelerated and converted into organic fertilizer with a fertilizer composition suitable for target crops. In particular, the fertilizer composition of the obtained organic fertilizer is adjustable by adjusting the composite feeding substrate. That is, the fertilizer composition of the organic fertilizer can be optimized according to different target crops. Therefore, the organic fertilizer formed by the method provided by the embodiments of the present disclosure does not need to undergo aerobic composting and adding animal and plant meal to adjust the fertilizer content, and can be directly applied to the soil for irrigation.

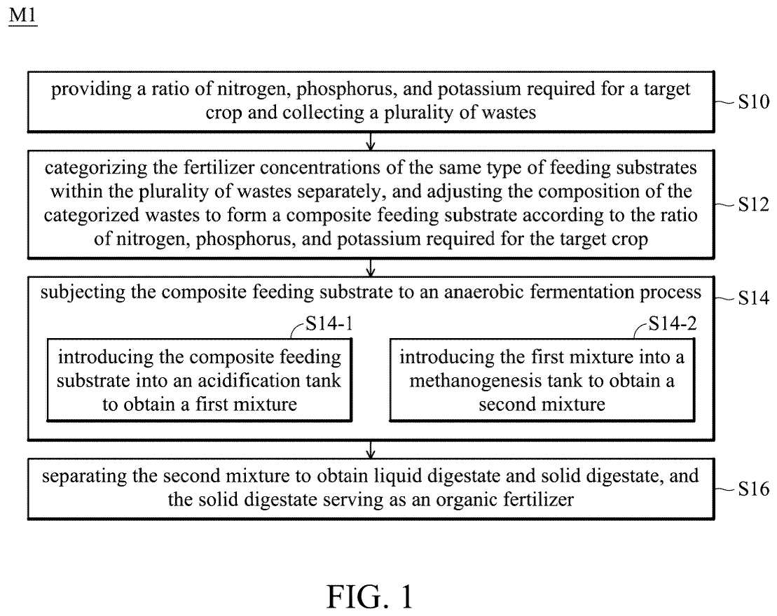

FIG. 1 shows a step flow chart of a method M1 of preparing an adjustable organic fertilizer in accordance with some embodiments of the present disclosure. It should be understood that, in accordance with some embodiments, additional steps may be added before, during and/or after the method M1 of preparing the adjustable organic fertilizer described below, or some steps may be replaced or omitted.

The method M1 of preparing the adjustable organic fertilizer includes step S10: providing a ratio of nitrogen, phosphorus, and potassium required for a target crop and collecting a plurality of wastes. Specifically, organic wastes containing nutrients required for the target crop (e.g., nitrogen, phosphorus, and potassium) may be collected, and appropriate organic waste types may be selected based on the fertilizer content requirements for the target crop.

The target crop may be any edible plant and/or ornamental plant. In accordance with some embodiments, the target crop may include vegetables, fruit trees/fruits, grains, legumes, root crops, plant raw materials, melons, prairie plants, lawns, spice plants, flowers, other suitable crops, or combinations thereof, but it is not limited thereto. In accordance with some embodiments, vegetables may include, for example, lettuce, green onions, bok choy, Chinese cabbage, large tomatoes, spinach, broccoli, onions, green peppers, etc., but they are not limited thereto. In accordance with some embodiments, fruit trees/fruits may include, for example, small-fruited tomatoes, apples, citrus fruits, pears, grapes, peaches, cherries, walnuts, almonds, bananas, strawberries, etc., but they are not limited thereto. In accordance with some embodiments, grains may include, for example, rice, barley, wheat, rye, oats, corn, sorghum, etc., but they are not limited thereto. In accordance with some embodiments, legumes may include, for example, soybeans, red beans, kidney beans, broad beans, peas, peanuts, etc., but they are not limited thereto. In accordance with some embodiments, root crops may include, for example, carrots, potatoes, sweet potatoes, radishes, lotus root, kohlrabi, etc., but they are not limited thereto. In accordance with some embodiments, plant raw materials may include, for example, cotton, hemp, rapeseed, sugar beet, hops, sugar cane, olives, rubber, coffee, tobacco, tea, etc., but they are not limited thereto. In accordance with some embodiments, melons may include, for example, pumpkin, cucumber, watermelon, cantaloupe, etc., but they are not limited thereto. In accordance with some embodiments, prairie plants may include, for example, duckweed, sorghum, cattail, clover, alfalfa, etc., but they are not limited thereto. In accordance with some embodiments, lawns may include, for example, Taipei grass, Conic grass, etc., but they are not limited thereto. In accordance with some embodiments, spice plants may include, for example, lavender, rosemary, thyme, basil, pepper, ginger, etc., but they are not limited thereto. In accordance with some embodiments, flowers may include, for example, chrysanthemums, roses, orchids, etc., but they are not limited thereto.

The waste may be waste containing organic matter. In accordance with some embodiments, the waste may include livestock waste, agricultural waste, food waste, another organic waste, or a combination thereof, but it is not limited thereto. In accordance with some embodiments, the livestock waste may include manure from poultry and livestock, such as pig manure, chicken manure, pig urine, chicken urine, or a combination thereof, but it is not limited thereto. In accordance with some embodiments, the agricultural waste may include biological agricultural waste, agricultural material waste, or a combination thereof, but it is not limited thereto. In accordance with some embodiments, the food waste may include raw kitchen waste, cooked kitchen waste, food waste from food factories, or a combination thereof, but it is not limited thereto.

Furthermore, the method M1 of preparing the adjustable organic fertilizer includes step S12: categorizing the fertilizer concentrations of the same type of feeding substrates within the wastes separately, and adjusting the composition of the categorized wastes to form a composite feeding substrate according to the ratio of nitrogen, phosphorus, and potassium required for the target crop. In accordance with some embodiments, the step of categorizing the fertilizer concentrations of the same type of feeding substrates within the various wastes may include analyzing the total nitrogen (N), total phosphoric anhydride (P₂O₅), and total potassium oxide (K₂O) concentrations of the same type of feeding substrates (for example, raw kitchen wastes, cooked kitchen wastes, Breeder chicken manure, layer chicken manure, meat chicken manure, black pig manure, or white pig manure) within the various wastes, but it is not limited thereto. In other words, in accordance with some embodiments, the total nitrogen, total phosphoric anhydride, and total potassium oxide in the wastes can be used as fertilizer content. In accordance with some embodiments, other nutrient components of the various wastes may also be analyzed, such as copper (Cu), zinc (Zn), sulfur (S), iron (Fe), manganese (Mn), boron (B), molybdenum (Mo), cobalt (Co), or other elements required for the target crop, but they are not limited thereto. Furthermore, in accordance with some embodiments, inductively coupled plasma optical emission spectroscopy (ICP-OES) may be used to analyze the total nitrogen, total phosphoric anhydride, and total potassium oxide components in the various wastes.

In addition, in accordance with some embodiments, the step of adjusting the composition of the categorized wastes to form the composite feeding substrate according to the ratio of nitrogen, phosphorus, and potassium required for the target crop may include adjusting the feed amounts of the various wastes through mass balance calculations, so that the concentrations of nitrogen, phosphorus, and potassium in the composite feeding substrate are higher than the concentrations required for the target crop. Specifically, considering that during the subsequent anaerobic fermentation metabolic processes, microorganisms will utilize nitrogen, phosphorus, and potassium as nutrients for cell growth, and to counteract the potassium ion loss caused by acidification and proton imbalance due to high concentrations of ammonia nitrogen in the environment, the fertilizer concentrations in the composite feeding substrate are adjusted to be higher than the concentrations required for the target crop.

In accordance with some embodiments, the concentrations of nitrogen, phosphorus, and potassium of the composite feeding substrate are 1 to 4 times, or 2 to 3 times, higher than the concentrations required for the target crop. Furthermore, in accordance with some embodiments, the concentration ratio of nitrogen to phosphorus to potassium in the composite feeding substrate may be in a range of 1 to 6:1 to 6:1 to 6, but it is not limited thereto. The concentration ratio of nitrogen to phosphorus to potassium in the composite feeding substrate can be adjusted corresponding to different target crops.

For example, in accordance with some embodiments, the target crop is green onions, and the fertilizer concentration ratio (nitrogen:phosphorus:potassium (vol %)) required for green onions is approximately 2:1:1. The feed amounts of various wastes may be adjusted through mass balance calculations so that the fertilizer concentration ratio (nitrogen:phosphorus:potassium (vol %)) in the composite feeding substrate is approximately 3 to 5:1 to 3:1 to 3, such as 3:1:1, 3:1:2, 3:2:2, 4:2:2, 4:2:3, 4:3:3, 5:2:3, or 5:3:3, but it is not limited thereto. In accordance with some other embodiments, the target crop is lettuce, and the fertilizer concentration ratio (nitrogen:phosphorus:potassium (vol %)) required for lettuce is approximately 2:2:1. The feed amounts of various wastes may be adjusted through mass balance calculations so that the fertilizer concentration ratio (nitrogen:phosphorus:potassium (vol %)) in the composite feeding substrate is approximately 2 to 5:2 to 5:2 to 4, such as 2:2:2, 2:3:3, 3:3:2, 3:3:3, 3:4:2, 4:3:2, 4:3:3, 4:4:3, 4:5:3, 5:4:3, or 5:5:3, but it is not limited thereto. In accordance with some other embodiments, the target crop is bok choy, and the fertilizer concentration ratio (nitrogen:phosphorus:potassium (vol %)) required for bok choy is approximately 4:3:3. The feed amounts of various wastes may be adjusted through mass balance calculations so that the fertilizer concentration ratio (nitrogen:phosphorus:potassium (vol %)) in the composite feeding substrate is approximately 2 to 6:2 to 6:2 to 6, such as 5:4:4, but it is not limited thereto. In accordance with some other embodiments, the target crop is tomatoes, and the fertilizer concentration ratio (nitrogen:phosphorus:potassium (vol %)) required for tomatoes is 3:2:2. The feed amounts of various wastes may be adjusted through mass balance calculations so that the fertilizer concentration ratio (nitrogen:phosphorus:potassium (vol %)) in the composite feeding substrate is approximately 2 to 5:2 to 5:2 to 5, such as 4:3:3, but it is not limited thereto.

Furthermore, the method M1 of preparing the adjustable organic fertilizer includes step S14: subjecting the composite feeding substrate to an anaerobic fermentation process. Specifically, the anaerobic fermentation process may include step S14-1: introducing the composite feeding substrate into an acidification tank to obtain a first mixture, and step S14-2: introducing the first mixture into a methanogenesis tank to obtain a second mixture. The anaerobic fermentation process can be used to process composite feeding substrates to produce solid digestate, liquid digestate and biogas, and the biogas includes methane. Specifically, the anaerobic fermentation process can decompose and transform small molecular organic matter through the biochemical metabolism of microorganisms to produce solid digestate, liquid digestate and biogas. The anaerobic fermentation process may be carried out through a high-temperature anaerobic co-digestion system including an acidification tank and a methanogenesis tank.

The acidification tank includes acidifying bacteria, and the pH value of the acidification tank may be between 3 and 7.5, and the temperature of the acidification tank may be between 30° C. and 60° C. In accordance with some embodiments, the reaction pH value of the acidification tank may be pH 3.5, pH 4, pH 4.5, pH 5, pH 5.5, pH 6, pH 6.5 or pH 7, but it is not limited thereto. In accordance with some embodiments, the acidifying bacteria may include Defluviitoga, Thermoanaerobacterales, Syntrophomonadaceae, Synergistaceae, Limnochordales, Tepidimicrobium, Symbiobacterium, Syntrophaceticus, another suitable acidifying bacteria or a combination thereof, but it is not limited thereto.

In accordance with some embodiments, the reaction temperature of the acidification tank may be between 35° C. and 55° C., for example, 40° C., 45° C. or 50° C., but it is not limited thereto. Moreover, in accordance with some embodiments, the capacity of the acidification tank may be between 5 liters and 10 liters, for example, 6 liters, 7 liters, 8 liters or 9 liters, but it is not limited thereto. In accordance with some embodiments, the hydraulic retention time (HRT) of the acidification tank may be between 3 days and 10 days, for example, 4 days, 5 days, 6 days, 7 days, 8 days or 9 days, but it is not limited thereto.

The methanogenesis tank includes methanogens, and the pH value of the methanogenesis tank may be between 6 and 8, and the temperature of the methanogenesis tank may be between 30° C. and 60° C. In accordance with some embodiments, the reaction pH value of the methanogenesis tank may be pH 6.5, pH 7, or pH 7.5, but it is not limited thereto. In accordance with some embodiments, the reaction temperature of the methanogenesis tank may be between 35° C. and 55° C., for example, 40° C., 45° C. or 50° C., but it is not limited thereto. In accordance with some embodiments, the methanogens may include Methanoculleus, Methanospirillum, Methanothermobacter, Methanosarcina, and other suitable methanogen species, or a combination thereof, but it is not limited thereto. Furthermore, in accordance with some embodiments, the capacity of the methanogenesis tank may be between 10 liters and 20 liters, for example, 11 liters, 12 liters, 13 liters, 14 liters, 15 liters, 16 liters, 17 liters, 18 liters or 19 liters, but it is not limited thereto. In accordance with some embodiments, the hydraulic retention time of the methanogenesis tank may be between 15 days and 25 days, for example, 16 days, 17 days, 18 days, 19 days, 20 days, 21 days, 22 days, 23 days, or 24 days, but it is not limited thereto.

In accordance with some embodiments, the dominant acidifying bacteria in the anaerobic co-digestion system may include Defluviitoga, Thermoanaerobacterales, Syntrophomonadaceae, Synergistaceae, Limnochordales, Tepidimicrobium, Symbiobacterium, Syntrophaceticus, another suitable acidifying bacteria or a combination thereof, but it is not limited thereto. In accordance with some embodiments, the proportion of dominant acidifying bacteria is about 35% to 55% of the microbial community, for example, 40%, 45% or 50%, but it is not limited thereto.

In accordance with some embodiments, the dominant methanogens in the anaerobic co-digestion system include Methanoculleus, Methanospirillum, Methanothermobacter, or Methanosarcina. In accordance with some embodiments, the proportion of dominant methanogens is about 5% to 10% of the microbial community, for example, 6%, 7%, 8% or 9%, but it is not limited thereto.

It is noteworthy that as the rates of microbial hydrolysis, acidification, and methane production increase, high concentrations of ammonia nitrogen are released, which can inhibit the metabolic pathways of anaerobic microorganisms. However, the acidifying bacteria and methanogens used in the embodiments of the present disclosure possess characteristics that allow them to tolerate high concentrations of ammonia nitrogen. This capability can improve the rate of anaerobic fermentation, thereby accelerating the stabilization and maturation of organic matter and converting it into organic fertilizer with a fertilizer composition suitable for the target crop.

In accordance with some embodiments, through the configuration of the aforementioned anaerobic fermentation process, the total solid content (TS) of the feed (composite feeding substrate) can be increased. Specifically, in accordance with some embodiments, the total solid content of the composite feeding substrate may be between 5% and 15%, for example, 6%, 7%, 8%, 9%, 10%, 11%, 12%, 13%, 14% or 15%, but it is not limited thereto. Moreover, in accordance with some embodiments, the ammonia nitrogen concentration of the composite feeding substrate may be between 800 mg/L and 4000 mg/L, or between 900 mg/L and 3000 mg/L, or between 1000 mg/L and 2500 mg/L, for example, 1100 mg/L, 1200 mg/L, 1300 mg/L, 1400 mg/L, 1500 mg/L, 1600 mg/L, 1700 mg/L, 1800 mg/L, 1900 mg/L, 2000 mg/L, 2100 mg/L, 2200 mg/L, 2300 mg/L or 2400 mg/L, but it is not limited thereto.

In accordance with some embodiments, the volatile suspended solids (VSS) removal rate of the aforementioned high-temperature anaerobic co-digestion system can reach more than 60%, for example, more than 65%, more than 70% or more than 75%. In accordance with some embodiments, the chemical oxygen demand (COD) removal rate of the aforementioned high-temperature anaerobic co-digestion system can reach more than 60%, for example, more than 65%, more than 70% or more than 75%.

Next, the method M1 of preparing the adjustable organic fertilizer includes step S16: separating the second mixture to obtain liquid digestate and solid digestate, and the solid digestate serving as an organic fertilizer. Specifically, the products of the anaerobic fermentation process can be separated into solid, liquid, and gas to obtain solid digestate, liquid digestate, and biogas. In accordance with some embodiments, the solid digestate may be subjected to further concentration and dehydration steps. In accordance with some embodiments, the biogas may be subjected to a biological desulfurization step to obtain methane, which can be used as natural gas to generate electricity.

It is noteworthy that the solid digestate obtained by the aforementioned separation of the second mixture can be used as organic fertilizer without further fertilizer content adjustment. The solid digestate does not need to undergo aerobic composting or adding animal and plant meal to adjust the fertilizer content, and can be directly applied to the soil for irrigation. In other words, the method M1 of preparing the adjustable organic fertilizer may not include an aerobic fermentation step. Furthermore, in accordance with some embodiments, the carbon/nitrogen (C/N) ratio of the obtained organic fertilizer may be between 10 and 20, for example, 11, 12, 13, 14, 15, 16, 17, 18 or 19, but it is not limited thereto. The carbon/nitrogen (C/N) ratio of the obtained organic fertilizer meets the requirements of the Fertilizer Management Act (C/N<20). In accordance with some embodiments, the fertilizer content of the organic fertilizer is substantially less than the fertilizer content of the various wastes. For example, the fertilizer content of the various wastes and the organic fertilizer all includes total nitrogen, total phosphoric anhydride, and total potassium oxide, and the ratio of total nitrogen:total phosphoric anhydride:total potassium oxide of the organic fertilizers is smaller than the ratio of total nitrogen:total phosphoric anhydride:total potassium oxide of the various wastes.

In order to make the above and other objects, features, and advantages of the present disclosure more comprehensible, several embodiments and comparative examples are listed below for detailed description. However, these examples are not intended to limit the scope of the present disclosure.

### Example 1—Characteristics Analysis of Material Source

Firstly, agricultural waste (such as crushed raw kitchen waste and cooked kitchen waste) and livestock manure (such as pig manure, chicken manure, pig urine, and chicken urine) were collected as raw materials for the composite feeding substrate. Subsequently, an inductively coupled plasma optical emission spectroscopy (ICP-OES) was used to analyze the components of the aforementioned wastes. The analysis results are shown in Table 1 below. The unit percentages (%) used in Table 1 are all percentages by volume (vol %).

TABLE 1

| | | Black | | White | | | | |
|---|---|---|---|---|---|---|---|---|
| | | pig's | Black pig's | pig's | White pig's | Breeder | Layer | Meat |
| | Agricultural | raw pig | urine | raw pig | urine | chicken | chicken | chicken |
| ICP-OES | waste | manure | wastewater | manure | wastewater | manure | manure | manure |
| TS (%) | 18 | 22 | | 42 | | 78 | 18 | 68 |
| Cu(mg/kg) | 13 | 17 | <1 | 17 | <1 | 48 | 76 | 55 |
| Zn(mg/kg) | 24 | 84 | <1 | 84.29 | <1 | 255 | 468 | 276 |
| N (%) | 3.15 | 3.63 | 0.18 | 1.86 | 0.3 | 1.73 | 1.69 | 2.32 |
| P (%) | 0.31 | 1.64 | 0.007 | 0.12 | 0.003 | 1.73 | 1.83 | 1.02 |
| K (%) | 1.62 | 0.27 | 0.04 | 0.07 | 0.01 | 2.45 | 2.71 | 2.50 |
| Total | 3.15 | 3.63 | 0.2 | 1.86 | 0.3 | 1.73 | 1.69 | 2.32 |
| nitrogen | | | | | | | | |
| (%) | | | | | | | | |
| Total | 0.70 | 3.77 | 0.015 | 0.29 | 0.007 | 3.96 | 4.20 | 2.34 |
| phosphoric | | | | | | | | |
| anhydride | | | | | | | | |
| (%) | | | | | | | | |
| Total | 1.95 | 0.32 | 0.05 | 0.10 | 0.01 | 2.95 | 3.27 | 3.02 |
| potassium | | | | | | | | |
| oxide | | | | | | | | |
| (%) | | | | | | | | |

Furthermore, the concentration of fertilizer content was confirmed using a soil NPK rapid test reagent. The agricultural waste was placed in a centrifuge tube, and an appropriate polymer flocculant was added for solid-water extraction. The clarified liquid from the upper layer of the centrifuge tube was poured into a colorimetric container. After adding reagent powder to the supernatant liquid and capping the container, it was vigorously shaken to dissolve the powder in the supernatant liquid. The color observed in the middle window of the color development box was matched to a color scale to quickly analyze the concentrations of nitrogen, phosphorus, and potassium fertilizers in the material source on-site. It should be noted that the rapid test reagent is a color development formula made in the laboratory. The purpose is to confirm the range of nitrogen, phosphorus, and potassium fertilizer content in the actual field, allowing for the determination of the amount of waste feed to facilitate subsequent adjustments. After developing the color scale with the laboratory-made formula, the precise concentrations of nitrogen, phosphorus, and potassium are determined using ICP-OES.

### Example 2—Setting Conditions for Anaerobic Fermentation Process and Performing Anaerobic Fermentation on Composite Feeding Substrates

The present disclosure evaluates the treatment efficiency of the anaerobic fermentation system under two different trial conditions, as specified in Table 2 below. As shown, the present disclosure establishes two trials: the acidification tank had a volume of 6 liters and a hydraulic retention time (HRT) of 5 days, while the methanogenesis tank had a volume of 12 liters and a hydraulic retention time of 20 days. Both the acidification tank and the methanogenesis tank were maintained at a high temperature of 55° C. for continuous reaction. The system was fed daily, and the pH value, temperature, and biogas production were recorded daily. Additionally, water quality analysis, solid/liquid digestate nutrient analysis, and microbial community analysis were conducted weekly.

TABLE 2

| Trial | | | | |
|---|---|---|---|---|
| Anaerobic | Trial 1 | | Trial 2 | |
| fermen- | | Methano- | | Methano- |
| tation | Acidification | genesis | Acidification | genesis |
| system | tank | tank | tank | tank |
| HRT (day) | 5 | 20 | 5 | 20 |
| Temperature | 55° C. | | | |
| Feeding | White pig | Effluent | Black pig | Effluent |
| substrates | manure (from | from | manure (from | from |
| | pig fed with | acidification | pig fed with | acidification |
| | feedstock) | tank | kitchen waste) | tank |
| | Breeder | | Breeder chicken | |
| | chicken manure | | manure | |
| | Agricultural | | Agricultural | |
| | waste | | waste | |
| | White pig urine | | Black pig urine | |
| | (from pig fed | | (from pig fed | |
| | with feedstock) | | with kitchen | |
| | | | waste) | |
| Feeding | 1 | 0.6 | 1 | 0.6 |
| volume | | | | |
| (liters/day) | | | | |

Based on the composition analysis of the aforementioned wastes, the fertilizer sources were categorized and the quantities for the anaerobic fermentation systems in Trial 1 and Trial 2 were calculated to form a composite feeding substrate.

Trial 1 (total solid content 7.5%): pig manure 4.8%, agricultural waste 2.1%, chicken manure 0.6%, the total feeding volume was 600 mL, of which the feeding volumes were pig manure 90 mL (15%), chicken manure 5 mL (0.8%), agricultural waste 85 mL (14%), pig manure and urine 420 mL (70%).

Trial 2 (total solid content 7.6%): pig manure 5.1%, agricultural waste 1.8%, chicken manure 0.7%, the total feeding volume was 600 mL, of which the feeding volumes were pig manure 140 mL (23%), chicken manure 5 mL (0.8%), agricultural waste 70 mL (11.2%), pig manure and urine 385 mL (64.2%).

The aforementioned total solid content is calculated by the following formula:

$$Total solid content = \frac{\begin{matrix}( solid content of A \times volume of A ) + \\ ( solid content of B \times volume of B ) + \\ ( solid content of C \times volume of A )\end{matrix}}{total feeding volume} ,$$

which A refers to pig manure, B refers to agricultural waste, and C refers to chicken manure.

In addition, the composition of the composite feeding substrate was analyzed using inductively coupled plasma optical emission spectroscopy (ICP-OES). The results are shown in Table 3 below. The unit percentages (%) used in Table 3 are all percentages by volume (vol %).

TABLE 3

| | Trial 1 | | | |
|---|---|---|---|---|
| | White | Breeder | | White pig |
| | pig | chicken | Agricultural | manure |
| Item | manure | manure | waste | and urine |
| Total solid content | 32 | 78 | 15 | 1 |
| (%) | | | | |
| Organic matter | 97.4 | 63.7 | 79.9 | — |
| content (%) | | | | |
| Total nitrogen (%) | 1.86 | 1.73 | 3.15 | 0.3 |
| Total phosphoric | 0.29 | 3.96 | 0.70 | 0.0148 |
| anhydride (%) | | | | |
| Total potassium | 0.09 | 2.95 | 1.95 | 0.0475 |
| oxide | | | | |
| (%) | | | | |
| Cu (mg/kg) | 17 | 48 | 13 | <1 |
| Zn (mg/kg) | 84 | 255 | 24 | <1 |
| | Trial 2 | | | |
| | Black | Breeder | | Black pig |
| | pig | chicken | Agricultural | manure |
| Item | manure | manure | waste | and urine |
| Total solid content (%) | 22 | 78 | 15 | 1 |
| Organic matter content | 90 | 63.7 | 79.9 | — |
| (%) | | | | |
| Total nitrogen (%) | 3.64 | 1.73 | 3.15 | 0.2 |
| Total phosphoric | 3.78 | 3.96 | 0.70 | 0.0068 |
| anhydride (%) | | | | |
| Total potassium oxide | 0.32 | 2.95 | 1.95 | 0.0121 |
| (%) | | | | |
| Cu (mg/kg) | 5 | 48 | 13 | <1 |
| Zn (mg/kg) | 23 | 255 | 24 | <1 |

As aforementioned, in Trial 1, the feeding substrates consisted of white pig manure (from pigs fed with feedstock), Breeder chicken manure, agricultural waste, and white pig urine (from pigs fed with feedstock), with a total solid content (TS) concentration of 7.5%, a ratio (vol %) of 4.8:0.6:2.1 (pig manure:chicken manure:agricultural waste), and an ammonia nitrogen concentration of 1000 mg/L. In Trial 2, the feeding substrates consisted of black pig manure (from pigs fed with kitchen waste), chicken manure, agricultural waste, and black pig urine (from pigs fed with kitchen waste), with a total solid content of 7.6%, a ratio (vol %) of 5.1:0.7:1.8 (pig manure:chicken manure:agricultural waste), and an ammonia nitrogen concentration of 2500 mg/L. The results shown in Table 3 indicate that compared to the feeding substrates derived from white pigs, the nitrogen, phosphorus, and potassium concentrations in the feeding substrates derived from black pigs are higher, approximately 2 to 3 times greater.

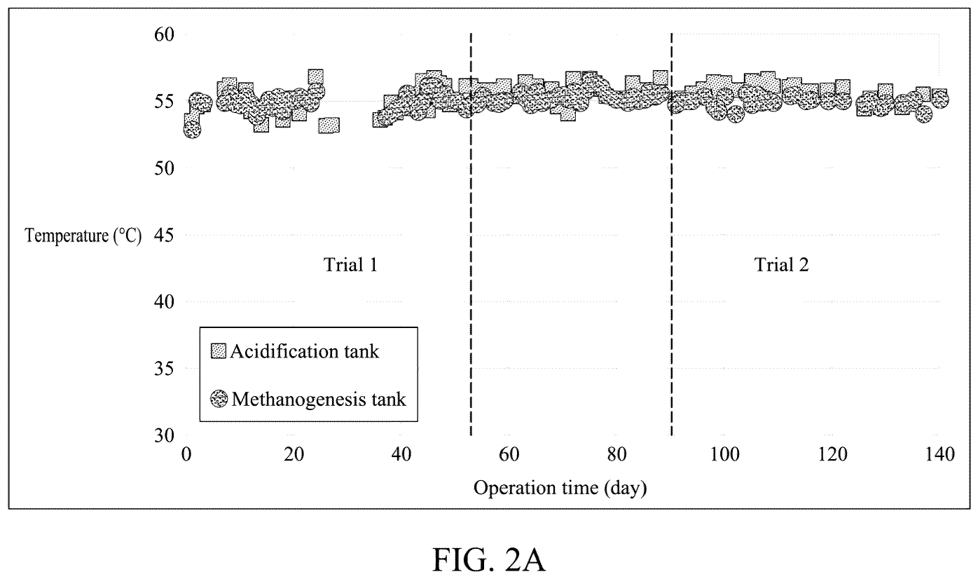

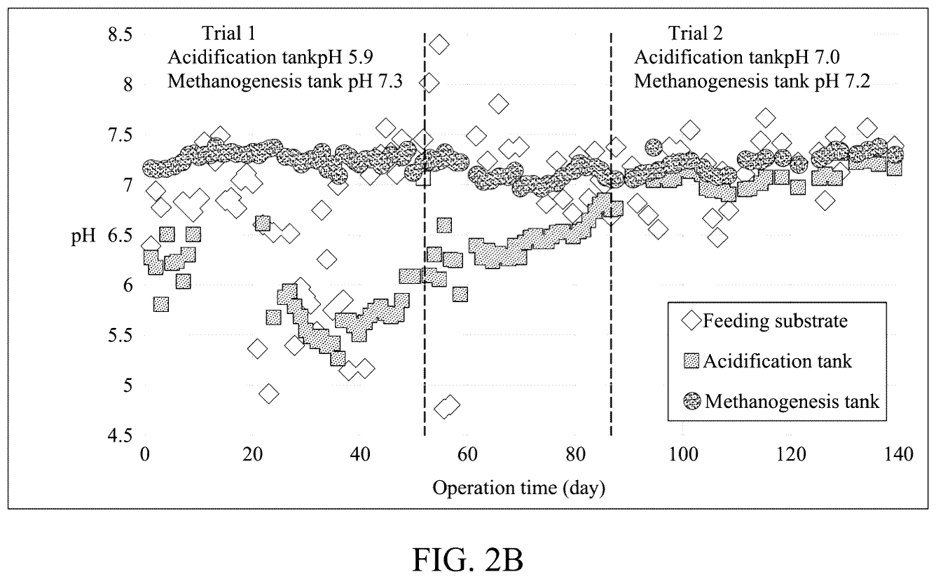

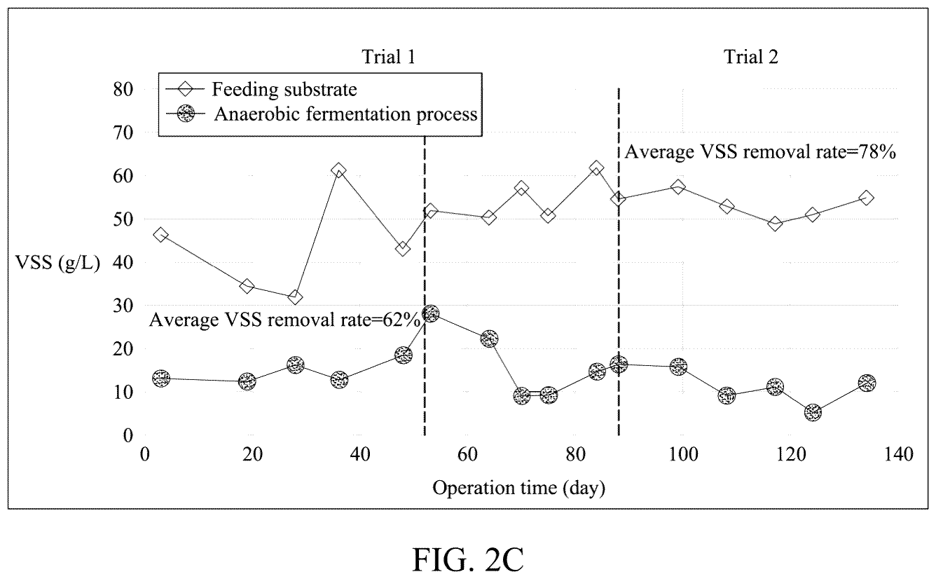

Moreover, FIG. 2A shows a schematic diagram of the temperature changes of the acidification tank and the methanogenesis tank during the anaerobic fermentation process. As shown in FIG. 2A, the average temperature of the acidification tank may be maintained at approximately 55.3° C., and the average temperature of the methanogenesis tank may be maintained at about 55° C. FIG. 2B shows a schematic diagram of the pH value changes of the acidification tank and the methanogenesis tank during the anaerobic fermentation process. As shown in FIG. 2B, the pH value of the methanogenesis tank may be maintained below approximately pH 7.3, demonstrating that the anaerobic fermentation system can buffer pH levels and reduce the inhibition of anaerobic biological metabolism by acidification and free ammonia. Moreover, FIG. 2C shows a schematic diagram of the concentration changes of volatile suspended solids (VSS) of the acidification tank and the methanogenesis tank during the anaerobic fermentation process. As shown in FIG. 2C, the VSS removal efficiencies of the anaerobic fermentation systems in Trial 1 and Trial 2 may be 62% and 78%, respectively, indicating that high concentrations of ammonia nitrogen may not inhibit microorganisms utilization of dissolved organic matter. Furthermore, next-generation sequencing (NGS) of the microbial community in the anaerobic fermentation system shows that dominant acidifying bacteria includes Defluviitoga, Thermoanaerobacterales, Syntrophomonadaceae, Synergistaceae, Limnochordales, Tepidimicrobium, Symbiobacterium, and Syntrophaceticus, constituting approximately 46% of the microbial community; dominant methanogens include Methanoculleus, Methanospirillum, and Methanothermobacter, constituting approximately 4% of the microbial community.

### Example 3—Forming Composite Feeding Substrate Based on Target Crop (Lettuce) and Performing Anaerobic Fermentation Process

Using lettuce as the target crop, the composition of the composite feeding substrate was adjusted to meet the required fertilizer concentration ratio (nitrogen:phosphorus:potassium (vol %)) being approximately 2:2:1). The fertilizer concentration in the composite feeding substrate was adjusted to exceed the concentration required for the target crop. Through mass balance calculations, the concentration ratio of nitrogen:phosphorus:potassium (vol %) of the composite feeding substrate was 3.23:3.13:3.17, with a concentration ratio of approximately 3:3:3. The composite feeding substrate was then subjected to anaerobic fermentation in Trial 2 (ammonia nitrogen concentration of 3000 mg/L), and the digestate was separated to obtain the solid digestate as organic fertilizer. Table 4 shows the composition analysis results of the composite feeding substrate, digestate, solid digestate and liquid digestate using inductively coupled plasma optical emission spectroscopy (ICP-OES). The unit percentages used in Table 4 are all percentages by volume (vol %).

TABLE 4

| | Trial 2 | | | |
|---|---|---|---|---|
| | Composite feeding | | Solid | Liquid |
| Item | substrate | Digestate | digestate | digestate |
| Total solid content (%) | 7.6 | 3 | 18 | 2 |
| Organic matter content | 70.2 | 69.2 | 93 | 68 |
| (%) | | | | |
| Total nitrogen (%) | 3.23 | 0.12 | 2.05 | 0.11 |
| Total phosphoric | 3.13 | 0.13 | 1.94 | 0.06 |
| anhydride (%) | | | | |
| Total potassium | 3.17 | 0.17 | 1.04 | 0.17 |
| peroxide (%) | | | | |
| Cu (mg/kg) | 2 | 2 | 8 | 2 |
| Zn (mg/kg) | 10 | 9 | 21 | 7 |

The analysis results show that nitrogen:phosphorus:potassium (vol %) of the obtained solid digestate is 2.05:1.94:1.04 with a concentration ratio of approximately 2:2:1, which can correspond to the fertilizer requirements for lettuce growth, making it suitable as an organic fertilizer.

### Example 4—Forming Composite Feeding Substrate Based on Target Crop (Green Onion) and Performing Anaerobic Fermentation Process

Using green onion as the target crop, the composition of the composite feeding substrate was adjusted to meet the required fertilizer concentration ratio (nitrogen:phosphorus:potassium (vol %)) being approximately 2:1:1). The composite feeding substrate was adjusted to exceed the required fertilizer concentration for the target crop, considering the types of microorganisms and metabolic energy needed during anaerobic fermentation. Through mass balance calculations, the concentration ratio of nitrogen:phosphorus:potassium (vol %) of the composite feeding substrate was 3.34:1.35:1.95, at a ratio of approximately 3:1:2. The composite feeding substrate was then subjected to anaerobic fermentation in Trial 1 (ammonia nitrogen concentration of 1000 mg/L), and the digestate was separated to obtain the solid digestate as organic fertilizer. Table 5 shows the composition analysis results of the composite feeding substrate, digestate, solid digestate and liquid digestate using ICP-OES. The unit percentages of Table 5 were all percentages by volume (vol %).

TABLE 5

| | Trial 1 | | | |
|---|---|---|---|---|
| | Composite feeding | | Solid | Liquid |
| Item | substrate | Digestate | digestate | digestate |
| Total solid content (%) | 6 | 4 | 16 | 3 |
| Organic matter content | 63.4 | 63.3 | 92.1 | 68.9 |
| (%) | | | | |
| Total nitrogen (%) | 3.24 | 0.12 | 2.85 | 0.07 |
| Total phosphoric | 1.35 | 0.11 | 1.05 | 0.11 |
| anhydride (%) | | | | |
| Total potassium oxide | 1.95 | 0.11 | 0.75 | 0.11 |
| (%) | | | | |
| Cu (mg/kg) | < 1 | 6 | 93 | 5 |
| Zn (mg/kg) | < 1 | 21 | 373 | 21 |

The analysis results show that nitrogen:phosphorus:potassium (vol %) of the obtained solid digestate is 2.85:1.05:0.75, with a concentration ratio of approximately 3:1:1, which can correspond to the fertilizer requirements for green onion growth, making it suitable as an organic fertilizer.

### Example 5—Pot Experiment for Target Crop (Lettuce)

The pH value and electrical conductivity of the solid digestate (organic fertilizer) obtained from Example 4 were measured, showing a pH value of 7.4 and an electrical conductivity (EC) of 3.6 mS/cm. Next, 5 g of the solid digestate sample was placed in a flask with 100 ml of distilled water at 75-80° C. After thorough stirring, the mixture was left to stand for 3 hours, and the leachate was filtered. 10 ml of the filtrate was placed in a petri dish with two layers of filter paper and 100 lettuce seeds. The petri dish was then incubated at 25° C. for about 4-5 days, and seeds that developed two complete cotyledons, roots, and root hairs were considered germinated. A control experiment was conducted using distilled water instead of filtrate. Each sample was tested in five replicates.

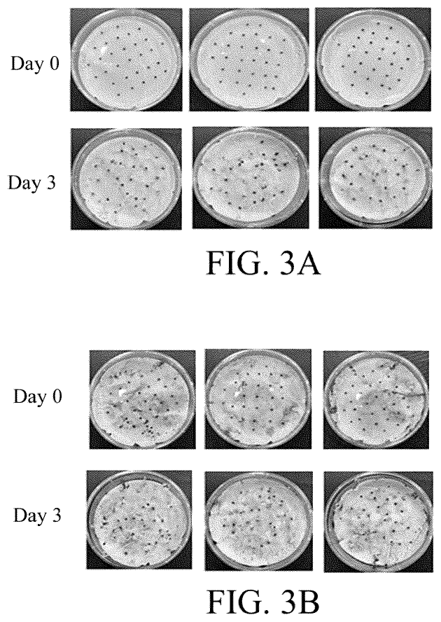

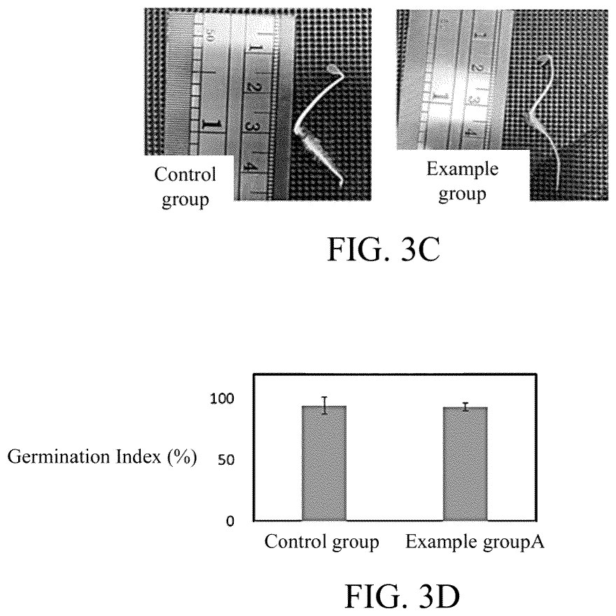

FIG. 3A and FIG. 3B respectively show the growth of seeds in the control group and the solid digestate sample group on the 0th and 3rd day. FIG. 3C shows the germination state of seeds in the control group and the solid digestate sample group. In addition, the seed germination rate and germination index (GI) of the control group and the solid digestate sample group on the 3rd day were calculated using the following formulas:

Seed Germination Rate=(Average number of germinated seeds in the solid digestate sample group/Average number of germinated seeds in the control group)×100%

Germination Index (%)=(Seed germination rate×Root length in the solid digestate sample group)/(Seed germination rate×Root length in the control group)×100%

The seed germination index is used to determine whether the solid digestate has fully fermented. Specifically, when the seed germination index is greater than 80%, it indicates that the solid digestate has thoroughly decomposed. As shown in FIG. 3D, the germination index of the solid digestate sample group (labeled as Example group in the figure) is greater than 80%, indicating complete maturation. Furthermore, as shown in FIG. 3C, it may be that the presence of some nutrients in the solid digestate effectively increased root length.

Furthermore, ICP-OES was also used to analyze the elemental composition of the solid digestate obtained in Example 4. The results are shown in Table 6 below. The miscellaneous compost items 5-11 refer to the specifications of miscellaneous compost fertilizers (item numbers 5-11) announced by the Ministry of Agriculture. As shown in Table 6, the residual amounts of organic matter, total nitrogen, total phosphorus anhydride, total potassium oxide, and heavy metals such as arsenic, cadmium, chromium, copper, mercury, nickel, lead, and zinc in the solid digestate were also analyzed. All items met the specified standards. The unit percentages of Table 6 were all percentages by volume (vol %).

TABLE 6

| | Nitrogen | Phosphorus | Potassium | Carbon | Sulfur | |
|---|---|---|---|---|---|---|
| Test items | (%) | (%) | (%) | (%) | (%) | |
| Solid digestate | 2.85 | 1.05 | 0.75 | 34.2 | 0.5 | |
| Miscellaneous | 0.6-5 | 0.3-6 | 0.3-4 | — | — | |
| compost fertilizers | | | | | | |
| 5-11 | | | | | | |
| | Calcium | Magnesium | Manganese | Iron | Copper | |
| Test items | (ppm) | (ppm) | (ppm) | (ppm) | (ppm) | pH |
| Solid digestate | 23733.5 | 5565.64 | 457.35 | 27130.4 | 74.09 | 7.4 |
| Miscellaneous | — | — | — | — | <100 | 5-9 |
| compost fertilizers | | | | | | |
| 5-11 | | | | | | |
| | Zinc | Sodium | Cadmium | Chromium | Nickel | |
| Test items | (ppm) | (ppm) | (ppm) | (ppm) | (ppm) | |
| Solid digestate | 149.96 | 2308.42 | 0.51 | 42.22 | 15.82 | |
| Miscellaneous | <500 | <4% | <2 | <150 | <25 | |
| compost fertilizers | | | | | | |
| 5-11 | | | | | | |
| | Lead | Arsenic | Mercury | Organic | TOC | |
| Test items | (ppm) | (ppm) | (ppm) | matter (%) | (%) | C/N |
| Solid digestate | 5.9 | 14.89 | <0.01 | 72.6 | 27.2 | 12.1 |
| Miscellaneous | <150 | <25 | <1 | >50 | — | 10-20 |
| compost fertilizers | | | | | | |
| 5-11 | | | | | | |

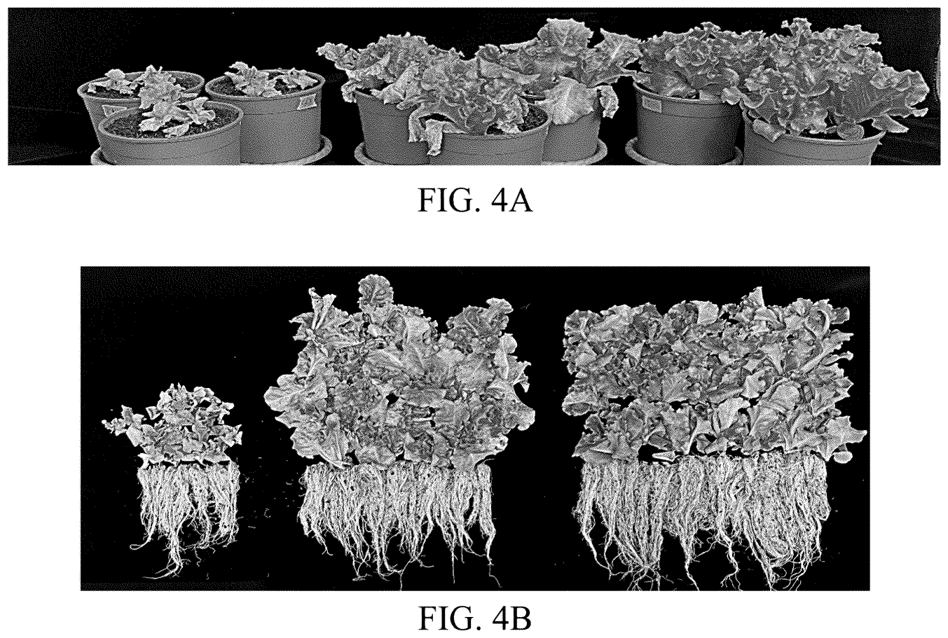

Subsequently, a three-month pot experiment was conducted using the solid digestate obtained from Example 4 as an organic fertilizer for lettuce. FIG. 4A and FIG. 4B show the growth results of lettuce after the three-month pot experiment. The left pot represented the control group cultivated with water. The middle pot represented the Comparative Example group cultivated with a commercially available organic fertilizer (Fwusow Green Fertilizer of Fwusow Industry Brand, a mixed organic fertilizer with an N:P:K ratio of 4:7:2). The right pot represented the Example group cultivated with the solid digestate from Example 4. As shown in FIG. 4A and FIG. 4B, the growth of both the above-ground and below-ground parts of the lettuce in the Example group was better compared to the control group and the Comparative Example group.

Furthermore, measurements of fresh weight, plant height, and chlorophyll content of the pot-grown lettuce were taken. The chlorophyll content was measured by a SPAD-502 chlorophyll meter (which determines the relative amount of chlorophyll in the leaf by measuring the transmittance of light through the leaf at two specific wavelengths). The results are shown in Table 7 below. Compared to the Comparative Example group with the commercially available organic fertilizer, the fresh weight of the above-ground part of the lettuce in the Example group increases by approximately 25%, the fresh weight of the below-ground part increases by approximately 33%, the plant height increases by approximately 23%, and the chlorophyll content increases by approximately 24%.

TABLE 7

| | | Comparative | |
|---|---|---|---|
| | | Example group | Example group |
| | Control | (commercially available | (solid digestate |
| | group | organic fertilizer with | with |
| Item | (water) | N:P:K = 4:7:2) | N:P:K = 2:2:1) |
| Fresh weight of | 8.30 ± | 84.33 ± 6.54^{b} | 105.83 ± 4.41^{c} |
| above-ground part | 0.29^{a} | | |
| (g) | | | |
| Fresh weight of | 6.17 ± | 38.97 ± 1.09^{b} | 51.93 ± 8.22^{c} |
| below-ground part | 0.34ª | | |
| (g) | | | |
| Plant height (cm) | 3.00 ± | 7.87 ± 0.21^{b} | 9.70 ± 0.21^{c} |
| | 0.34ª | | |
| Chlorophyll content | 6.28 ± | 16.63 ± 1.05^{b} | 20.60 ± 0.08^{c} |
| SPAD | 1.34ª | | |

*The differences between treatments were analyzed using one-way analysis of variance (ANOVA).
The significant differences between the means (P < 0.05) were determined using the least significant difference (LSD) test.

### Example 6—Pot Experiment for Target Crop (Green Onions)

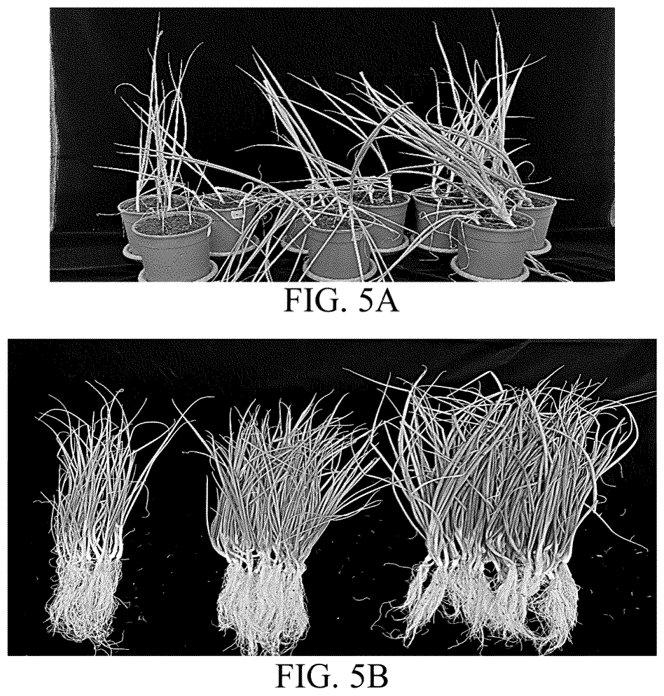

A three-month pot experiment was conducted using the solid digestate obtained from Example 5 as an organic fertilizer for green onions. FIG. 5A and FIG. 5B show the results of the pot experiments of green onion after three months of the pot experiment. The left pot represented the control group cultivated with water. The middle pot is the Comparative Example group cultivated with a commercially available organic fertilizer (Fwusow Green Fertilizer of Fwusow Industry Brand, a mixed organic fertilizer with an N:P:K ratio of 4:7:2). The right pot is the Example group cultivated with the solid digestate from Example 4. As shown in FIG. 5A and FIG. 5B, compared to the control group and the Comparative Example group, the growth of both above-ground and below-ground growth of the green onions in the Example group is better.

Furthermore, the number of leaves, plant height, and weight of the potted green onions were measured, with the results shown in Table 8 below. Compared to the Comparative Example group using the commercially available organic fertilizer, the number of leaves in the Example group increased by approximately 31%, the plant height increased by approximately 21%, and the weight increased by approximately 110%.

TABLE 8

| | | Comparative | |
|---|---|---|---|
| | | Example group | Example |
| | | (commercially | group |
| | | available organic | (solid digestate |
| | Control group | with fertilizer | with |
| Item | (water) | N:P:K = 4:7:2) | N:P:K = 2:1:1) |
| Leave | 3.19 ± 0.74ª | 5.89 ± 0.24a^{b} | 7.70 ± 0.14^{b} |
| number | | | |
| Plant | 27.70 ± 8.71ª | 44.24 ± 0.44^{b} | 53.53 ± 0.91^{b} |
| height (cm) | | | |
| Weight (g) | 21.33 ± 13.89ª | 73.33 ± 8.06^{b} | 154.33 ± 9.88^{c} |

*The differences between treatments were analyzed using one-way analysis of variance.
The significant differences between the means (P < 0.05) were determined using the least significant difference (LSD) test.

### Example 7—Conditions Setting for Anaerobic Fermentation Process and Anaerobic Fermentation of Composite Feeding Substrates

Similar to the method described in Example 2, two additional trials were established, with the conditions set as shown in Table 9 below. The volume of acidification tank was 6 liters with a hydraulic retention time (HRT) of 5 days, and the volume of methanogenesis tank was 12 liters with a hydraulic retention time of 20 days. Both the acidification tank and the methanogenesis tank were maintained at a high temperature of 55° C. for continuous reactions. The system was fed daily, and pH value, temperature, and biogas production were recorded daily. Additionally, water quality analysis, solid/liquid digestate nutrient analysis, and microbial community analysis were conducted weekly. In this example, the feeding substrates also included kitchen waste.

TABLE 9

| Trial | Trial 3 | | Trial 4 | |
|---|---|---|---|---|
| Anaerobic | | Methano- | | Methano- |
| fermentation | Acidification | genesis | Acidification | genesis |
| system | tank | tank | tank | tank |
| HRT (day) | 5 | 20 | 5 | 20 |
| Temperature | 55° C. | | | |
| Feeding | White pig | Effluent | Black pig | Effluent |
| substrates | manure (from | from | manure (from | from |
| | pig fed with | acidification | pig fed with | acidification |
| | feedstock) | tank | kitchen waste) | tank |
| | Kitchen waste | | Kitchen waste | |
| | Agricultural | | Breeder chicken | |
| | waste | | manure | |
| | White pig urine | | Agricultural | |
| | (from pig fed | | waste | |
| | with feedstock) | | Black pig urine | |
| | | | (from pig fed | |
| | | | with kitchen | |
| | | | waste) | |
| Feeding | 1 | 0.6 | 1 | 0.6 |
| volume | | | | |
| (liters/day) | | | | |

Based on the composition analysis of the aforementioned wastes, the fertilizer sources were categorized and the quantities for the anaerobic fermentation systems in Trial 3 and Trial 4 were calculated to form a composite feeding substrate.

Trial 3 (total solid content 7.2%): pig manure 32.23%, kitchen waste 10%, agricultural waste 10.14%, pig urine 1%, the total feeding volume was 600 mL, of which the feeding volumes were pig manure 100 mL (5.4%), kitchen waste 40 mL (1.2%), and agricultural waste 70 mL (0.7%).

Trial 4 (total solid content 7.2%): pig manure 22%, kitchen waste 10%, chicken manure 71%, agricultural waste 10.14%, pig urine 1%, the total feeding volume was 600 mL, of which the feeding volumes were pig manure 120 mL (4.4%), kitchen waste 50 mL (0.8%), chicken manure 5 mL (0.8%), chicken manure 5 mL (0.6%), and agricultural waste 10 mL (1.4%).

In addition, the composition of the composite feeding substrate was analyzed by inductively coupled plasma optical emission spectroscopy (ICP-OES). The results are shown in Table 10 below. The unit percentages (%) of Table 10 are all percentages by volume (vol %).

TABLE 10

| | Trial 3 | | | | |
|---|---|---|---|---|---|
| | Black | | Agricul- | Black pig | |
| | pig | Chicken | tural | manure and | |
| Item | manure | waste | waste | urine | |
| Total solid | 22 | 1 | 10.1 | 1 | |
| content (%) | | | | | |
| Organic matter | 90 | 98 | 79.9 | — | |
| content (%) | | | | | |
| Total nitrogen | 3.64 | 0.5 | 3.15 | 0.2 | |
| (%) | | | | | |
| Total phosphoric | 3.28 | 1.91 | 0.7 | 0.0068 | |
| anhydride (%) | | | | | |
| Total potassium | 0.32 | 3.73 | 1.95 | 0.0121 | |
| oxide | | | | | |
| (%) | | | | | |
| Cu (mg/kg) | 5 | — | 13 | <1 | |
| Zn (mg/kg) | 23 | — | 24 | <1 | |
| | Trial 4 | | | | |
| | Black | Breeder | | Agricul- | Black pig |
| | pig | chicken | Kitchen | tural | manure and |
| Item | manure | manure | waste | waste | urine |
| Total solid content | 22 | 10 | 1 | 10.1 | 1 |
| (%) | | | | | |
| Organic matter | 90 | 63.7 | 98 | 79.9 | — |
| content (%) | | | | | |
| Total nitrogen (%) | 3.64 | 1.73 | 0.5 | 3.15 | 0.2 |
| Total phosphoric | 3.28 | 3.96 | 1.91 | 0.7 | 0.0068 |
| anhydride (%) | | | | | |
| Total potassium | 0.32 | 2.95 | 3.73 | 1.95 | 0.0121 |
| oxide | | | | | |
| (%) | | | | | |
| Cu (mg/kg) | 5 | 48 | — | 13 | <1 |
| Zn (mg/kg) | 23 | 255 | — | 24 | <1 |

As aforementioned, in Trial 3, the feeding substrates consisted of white pig manure (from pigs fed with feedstock), kitchen waste, agricultural waste, and white pig urine (from pigs fed with feedstock), with a total solid content (TS) concentration of 7.2% and a ratio (vol %) of 5.4:1.2:0.7 (pig manure:kitchen waste:agricultural waste), with an ammonia nitrogen concentration of 1000 mg/L. In Trial 4, the feeding substrates consisted of black pig manure (from pigs fed with kitchen waste), kitchen waste, chicken manure, agricultural waste, and black pig urine (from pigs fed with kitchen waste), with a total solid content concentration of 7.2% and a ratio (vol %) of 4.4:0.8:0.6:1.4 (pig manure:kitchen waste:chicken manure:agricultural waste), with an ammonia nitrogen concentration of 2200 mg/L.

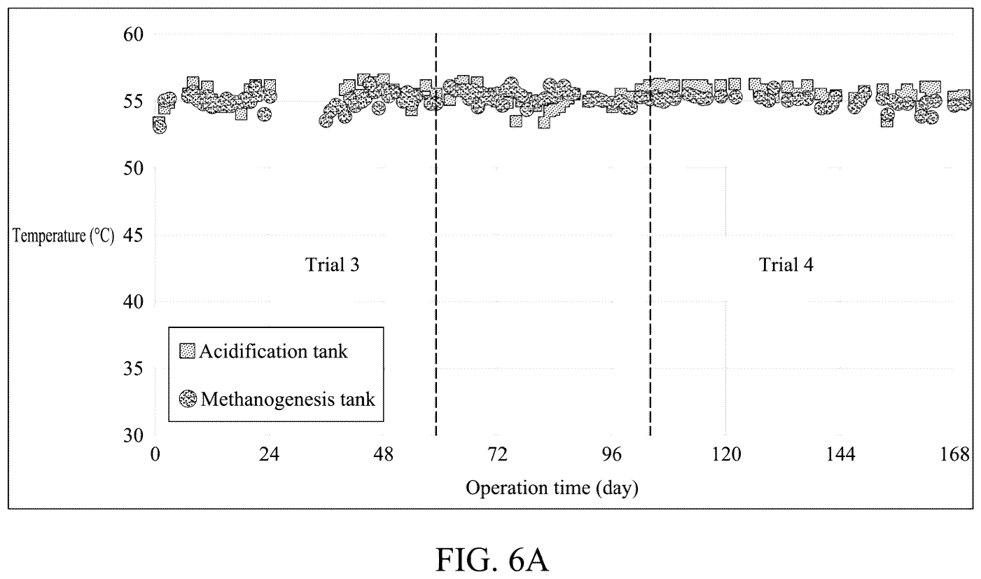

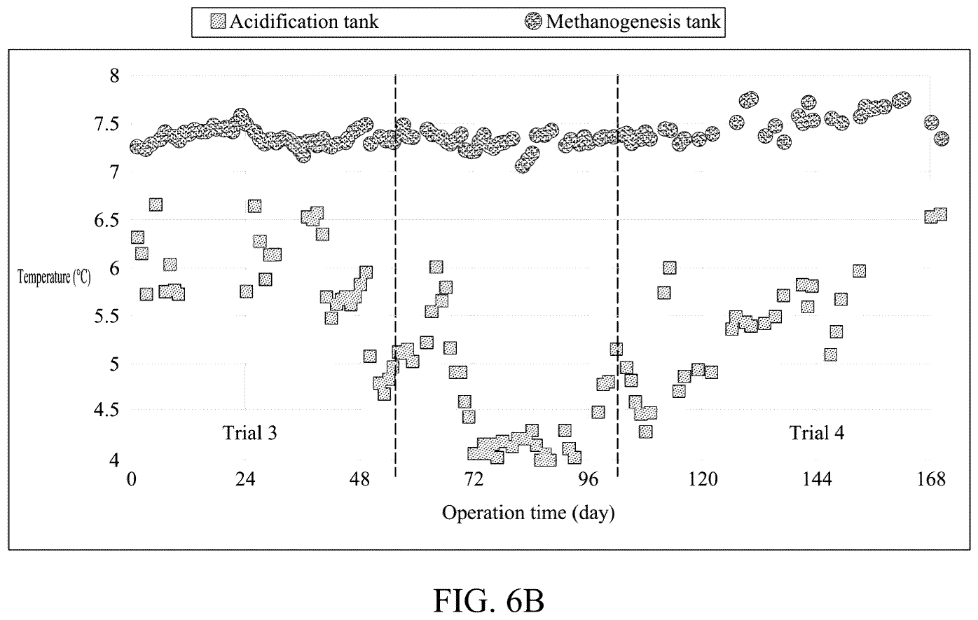

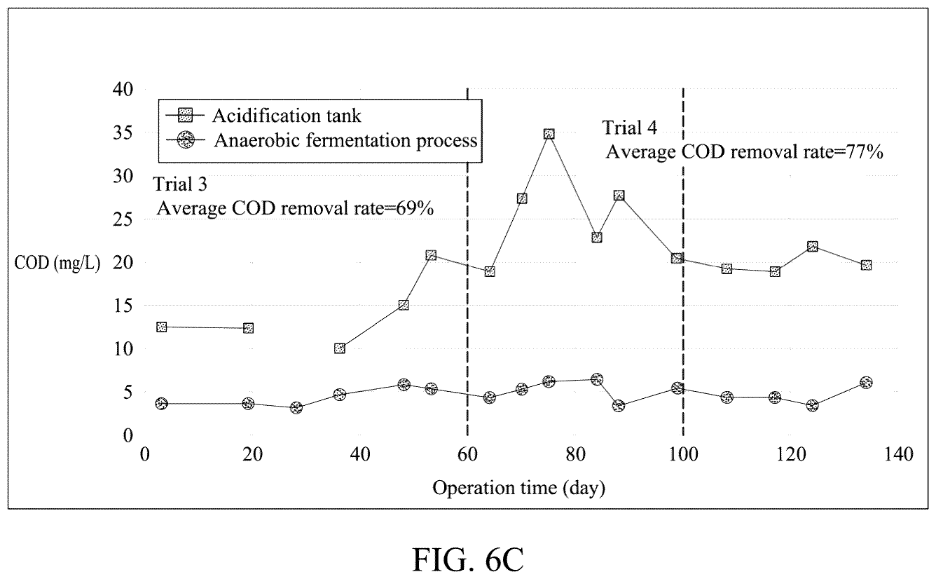

Furthermore, FIG. 6A shows a schematic diagram of the temperature changes of the acidification tank and the methanogenesis tank during the anaerobic fermentation process. As shown in FIG. 6A, the average temperature of the acidification tank may be maintained at around 55.3° C., and the average temperature of the methanogenesis tank may be maintained at around 55° C. FIG. 6B shows a schematic diagram of the pH value changes of the acidification tank and the methanogenesis tank during the anaerobic fermentation process. As shown in FIG. 6B, the pH value of the methanogenesis tank may be maintained below approximately pH 7.8, indicating that the anaerobic fermentation system may buffer pH levels and reduce the inhibition of anaerobic biological metabolism by acidification and free ammonia. Additionally, FIG. 6C shows a schematic diagram of the concentration changes of chemical oxygen demand (COD) during the anaerobic fermentation process. As shown in FIG. 6C, the COD removal efficiency of the anaerobic fermentation systems in Trial 3 and Trial 4 may reach 69% and 77%, respectively, indicating that high ammonia nitrogen concentrations may not inhibit the microbial utilization of dissolved organic matter.

Moreover, microbial community analysis of the anaerobic fermentation system using next-generation sequencing (NGS) shows that the dominant acidifying bacteria includes Defluviitoga, Thermoanaerobacterales, Syntrophomonadaceae, Synergistaceae, Limnochordales, Tepidimicrobium, Symbiobacterium, and Syntrophaceticus, constituting approximately 41% of the microbial community; dominant methanogens include Methanosarcina and Methanothermobacter, constituting approximately 7% of the microbial community.

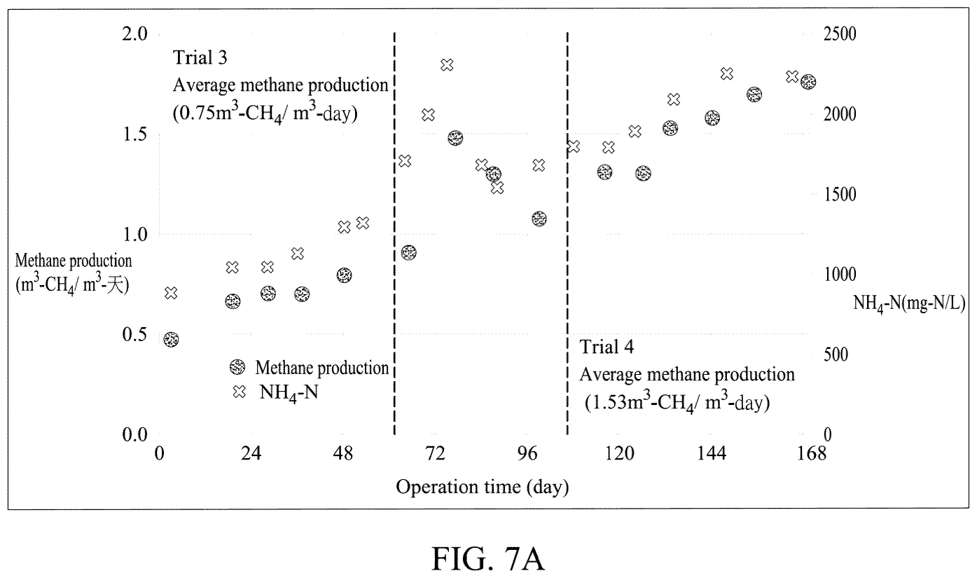

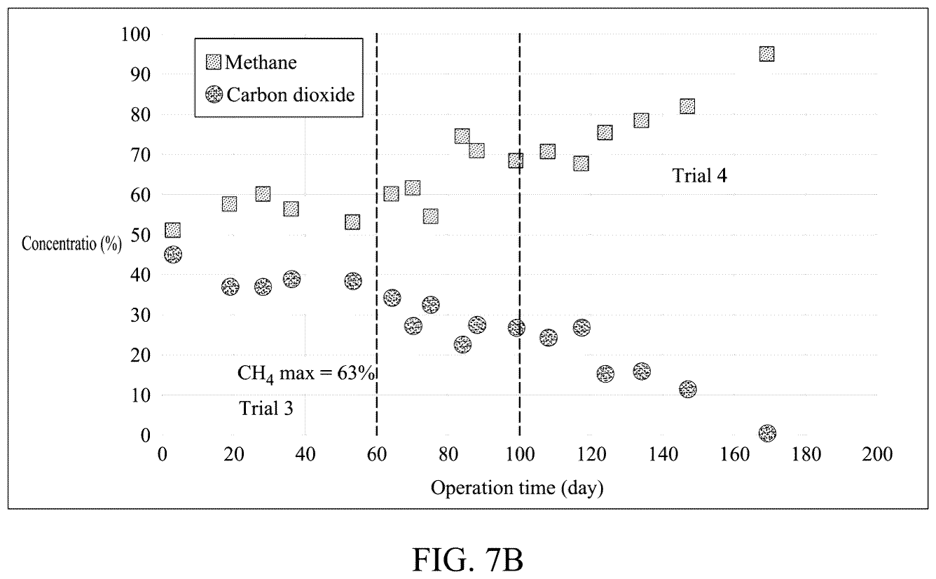

Furthermore, FIG. 7A shows a schematic diagram of changes in methane production during the anaerobic fermentation process, and FIG. 7B shows a schematic diagram of the concentration changes of methane and carbon dioxide during the anaerobic fermentation process. As shown in FIG. 7A, the methane production in Trial 3 reached 0.75 m³-CH₄/m³-day, while the methane production in Trial 4 reached 1.53 m³-CH₄/m³-day. As shown in FIG. 7B, under conditions of high ammonia nitrogen concentration, the average methane concentration could increase from 60% to 81%, and up to 95%.

### Example 8—Pot Experiment for Target Crop (Bok Choy)

The pH value and electrical conductivity (EC) of the solid digestate (organic fertilizer) obtained in Example 7 were measured by a pH meter and a conductivity meter, showing a pH value of 8 and an EC of 3.1 mS/cm. Next, 5 grams of the solid digestate sample were placed in an Erlenmeyer flask, and 100 ml of distilled water at 75-80° C. was added. After thorough stirring, the mixture was left to stand for 3 hours, and the leachate was filtered. Then, 10 ml of the filtrate was placed in a Petri dish containing two layers of filter paper and 100 seeds of bok choy. The Petri dish was then placed in a 25° C. incubator for about 4-5 days, and seeds that developed two complete cotyledons, roots, and root hairs were considered germinated. Additionally, a control experiment was conducted using distilled water instead of filtrate. Each sample was tested in five replicates.

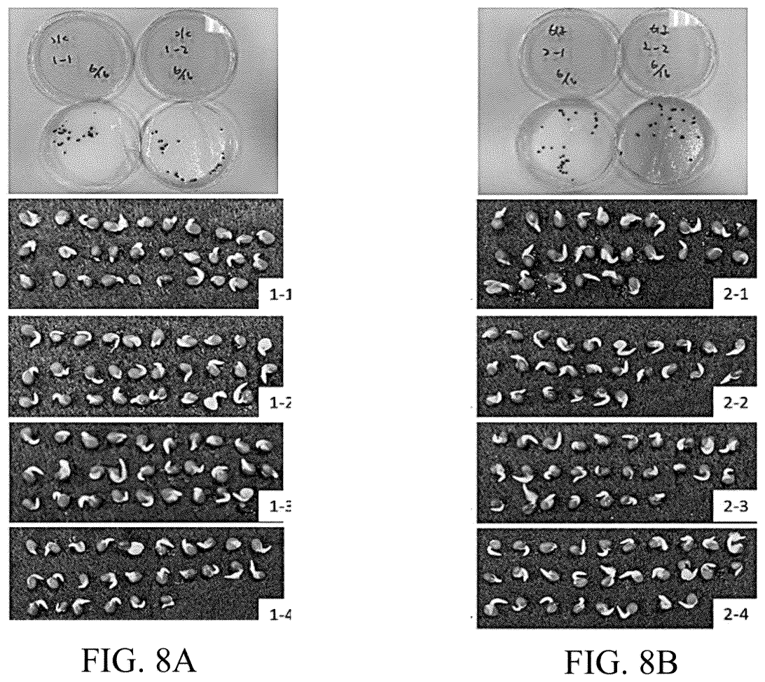

FIG. 8A and FIG. 8B respectively show the growth conditions of seeds in the control group and the solid digestate sample group on the 3rd day. Furthermore, the germination rate and germination index (GI) of the seeds in the control group and the solid digestate sample group on the 3rd day were calculated using the formula described in Example 5, confirming that the solid digestate had fully matured (germination index greater than 80%).

The elemental composition of the solid digestate obtained in Example 7 was analyzed using inductively coupled plasma optical emission spectrometry (ICP-OES), and the results are shown in Table 11. The miscellaneous compost items 5-11 refer to the specifications of miscellaneous compost fertilizers (item numbers 5-11) announced by the Ministry of Agriculture. As shown in Table 11, the residual amounts of organic matter, total nitrogen, total phosphorus anhydride, total potassium oxide, and heavy metals such as arsenic, cadmium, chromium, copper, mercury, nickel, lead, and zinc in the solid digestate were also analyzed. All items met the specified standards. The unit percentages of Table 11 are all percentages by volume (vol %).

TABLE 11

| | Organic | | | | Total | Total |
|---|---|---|---|---|---|---|
| | matter | | Nitrogen | Phosphorus | potassium | calcium |
| Test items | (%) | pH | (%) | (%) | oxide (%) | oxide (%) |
| Solid digestate | 73.1 | 8 | 4.0 | 3.0 | 3.0 | 9.5 |
| Miscellaneous | above 50 | 5~9 | 0.6~5 | 0.3~6 | 0.3~4 | — |
| compost | | | | | | |
| fertilizers 5-11 | | | | | | |

| | Total | Total | | | | |
|---|---|---|---|---|---|---|
| | magnesium | iron | Zinc | Sodium | Cadmium | Chromium |
| Test items | oxide (%) | (%) | (ppm) | (ppm) | (ppm) | (ppm) |
| Solid digestate | 0.5 | 0.2 | 302 | 0.5 | N.D. | 14 |
| Miscellaneous | — | — | <500 | <4% | <2 | <150 |
| compost | | | | | | |
| fertilizers 5-11 | | | | | | |

| | Nickel | Lead | Arsenic | Mercury | EC |
|---|---|---|---|---|---|
| Test items | (ppm) | (ppm) | (ppm) | (ppm) | (dS/m) |
| Solid digestate | 6.2 | 3 | N.D. | <0.01 | 3.1 |
| Miscellaneous | <25 | <150 | <25 | <1 | — |
| compost | | | | | |
| fertilizers 5-11 | | | | | |

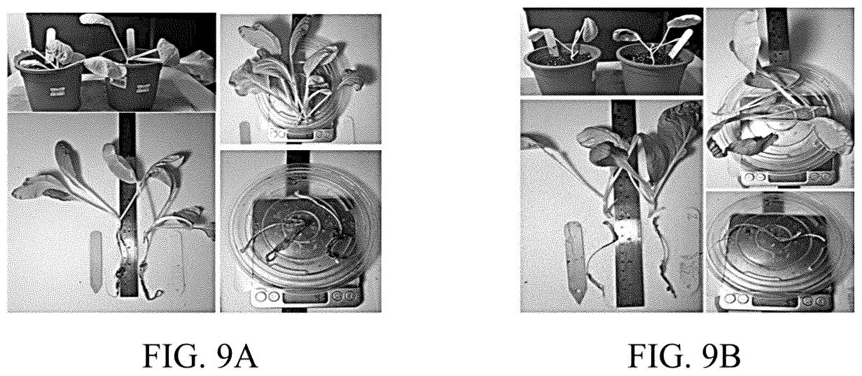

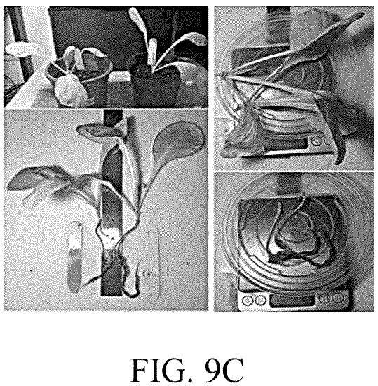

Subsequently, a three-month pot experiment was conducted using the solid digestate obtained from Example 7 as an organic fertilizer for bok choy. FIGS. 9A to 9C respectively show the growth results of bok choy in the Example group, Comparative Example group 1, and Comparative Example group 2 after the three-month pot experiment. The bok choy in Comparative Example 1 was cultivated using commercially available organic fertilizer (Fwusow Green Fertilizer of Fwusow Industry Brand, a mixed organic fertilizer with an N:P:K ratio of 4:7:2), while the bok choy in Comparative Example group 2 was cultivated using commercially available chemical fertilizer (nitrophosphorus-based Heiwangte No. 43 organic composite fertilizer of Known-You Brand, with a ratio of nitrogen:phosphorus anhydride:potassium oxide:magnesium oxide:calcium oxide:organic matter being 15:15:15:3:7.5:3). The bok choy in the Example group was cultivated using the solid digestate from Example 7. As shown in FIGS. 9A to 9C, the growth condition of the bok choy in the Example group was better (more leaves, greater plant height) compared to those in Comparative Example group 1 and Comparative Example group 2.

Further measurements of the fresh weight of the leaves, stems, and roots of the bok choy in the pots were conducted, and the results were shown in Table 12. Compared to the commercial organic fertilizer in Comparative Example group 1, the total fresh weight of the bok choy in the Example group increased by about 20%, and the total fresh weight of the bok choy in the Example group was comparable to that of the commercial chemical fertilizer in Comparative Example group 2.

TABLE 12

| | | Comparative | Comparative | Example |
|---|---|---|---|---|
| | | Example group | Example group 2 | group |
| | | 1 (commercially | (commercially | (solid |
| | | available | available | digestate |
| | Control | organic | chemical | with |
| | group | fertilizer with | fertilizer with | N:P:K = |
| Item | (water) | N:P:K = 4:7:2) | N:P:K = 1:1:1) | 4:3:3) |
| Fresh | 5.0 | 6.6 | 7.7 | 8.1 |
| weight of | | | | |
| leaves and | | | | |
| stems (g) | | | | |
| Fresh | 3.1 | 3.9 | 4.3 | 4.3 |
| weight of | | | | |
| roots (g) | | | | |
| Total | 8.1 | 10.5 | 12.0 | 12.4 |

### Example 9—Pot Experiment for Target Crop (Small-Fruited Tomato)

The solid digestate (organic fertilizer) for small-fruited tomatoes was obtained by a method similar to Example 7. The pH meter and conductivity measurements indicated that the solid digestate had a pH of 8 and an electrical conductivity (EC) of 2.3 mS/cm. Then, 5 g of the solid digestate sample was placed in an Erlenmeyer flask, 100 ml of distilled water at 75-80° C. was added, and the mixture was stirred and left to stand for 3 hours, and the leachate was filtered. Then, 10 ml of the filtrate was placed in a Petri dish containing two layers of filter paper and 100 seeds of small-fruited tomatoes. The Petri dish was then placed in a 25° C. incubator for about 4-5 days, and seeds that developed two complete cotyledons, roots, and root hairs were considered germinated. Additionally, each sample was tested in five replicates.

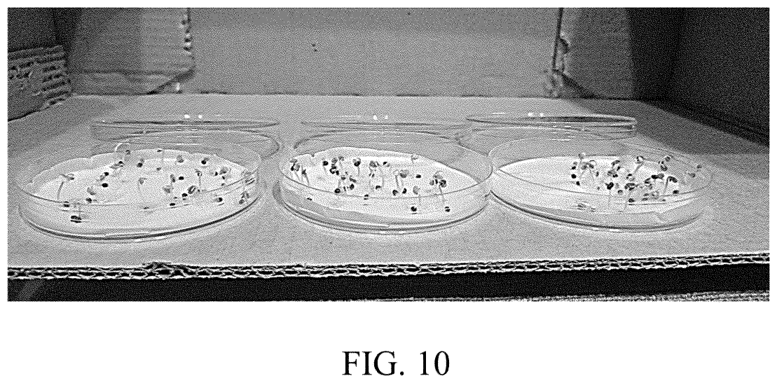

FIG. 10 shows the growth conditions of seeds in the solid digestate sample group on the 3rd day. In addition, the germination rate and germination index (GI) of the control group and the solid digestate sample group on the 3rd day were calculated using the formula described in Example 5, confirming that the solid digestate was fully matured (germination index greater than 80%).

The elemental composition of the solid digestate obtained in Example 7 was analyzed by inductively coupled plasma optical emission spectrometry (ICP-OES), and the results are shown in Table 13. The miscellaneous compost items 5-11 refer to the specifications of miscellaneous compost fertilizers (item numbers 5-11) announced by the Ministry of Agriculture. As shown in Table 13, the residual amounts of organic matter, total nitrogen, total phosphorus anhydride, total potassium oxide, and heavy metals such as arsenic, cadmium, chromium, mercury, nickel, lead, zinc in the solid digestate were also analyzed. All items met the specified standards. The unit percentages of Table 13 are all percentages by volume (vol %).

TABLE 13

| | Organic | | | | Total | Total |
|---|---|---|---|---|---|---|
| | matter | | Nitrogen | Phosphorus | potassium | calcium |
| Test items | (%) | pH | (%) | (%) | oxide (%) | oxide (%) |
| Solid digestate | 73.1 | 8 | 4.0 | 3.0 | 3.0 | 9.5 |
| Miscellaneous | 67 | 8 | 3.3 | 11.1 | 0.5 | 14 |
| compost | | | | | | |
| fertilizers 5-11 | | | | | | |

| | Total | Total | | | | |
|---|---|---|---|---|---|---|
| | magnesium | iron | Zinc | Sodium | Cadmium | Chromium |
| Test items | oxide (%) | (%) | (ppm) | (ppm) | (ppm) | (ppm) |
| Solid digestate | 0.5 | 0.2 | 302 | 0.5 | N.D. | 14 |
| Miscellaneous | 0.6 | 0.2 | 361 | 0.5 | N.D. | 12 |
| compost | | | | | | |
| fertilizers 5-11 | | | | | | |

| | Nickel | Lead | Arsenic | Mercury | EC |
|---|---|---|---|---|---|
| Test items | (ppm) | (ppm) | (ppm) | (ppm) | (dS/m) |
| Solid digestate | 6.2 | 3 | N.D. | <0.01 | 3.1 |
| Miscellaneous | 6.8 | 3 | N.D. | <0.01 | 2.3 |
| compost | | | | | |
| fertilizers 5-11 | | | | | |

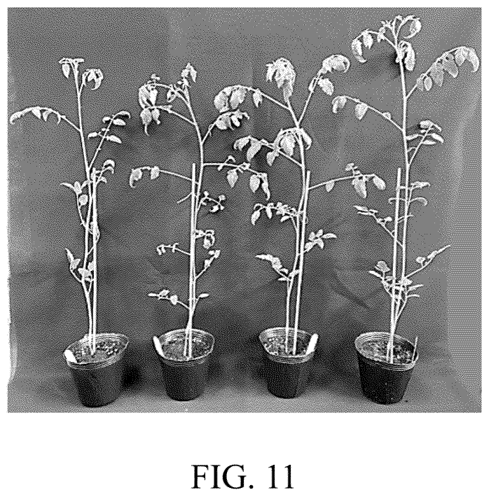

Subsequently, a pot experiment was conducted on small-fruited tomatoes using the aforementioned solid digestate as an organic fertilizer. FIG. 11, in which from left to right were the control group, Comparative Example group 1, Comparative Example group 2 and Example group. The potted plants in the control group were cultivated with water. The potted plants in the Comparative Example group 1 were cultivated with a commercially available chemical fertilizer group (nitrophosphorus-based Heiwangte No. 43 organic composite fertilizer of Known-You Brand, with a ratio of nitrogen:phosphorus anhydride:potassium oxide:magnesium oxide:calcium oxide:organic matter being 15:15:15:3:7.5:3). The potted plants in the Comparative Example group 2 were cultivated with a commercially available organic matter fertilizer group (Fwusow green Fertilizer of Fwusow Industry Brand, a mixed organic fertilizer with a ratio of nitrogen:phosphorus anhydride:potassium oxide being 4:7:2). The potted plants in the Example Group are cultivated with the solid digestate corresponding to small-fruited tomatoes (with the ratio of nitrogen to phosphorus anhydride to potassium oxide being 3:2:2).

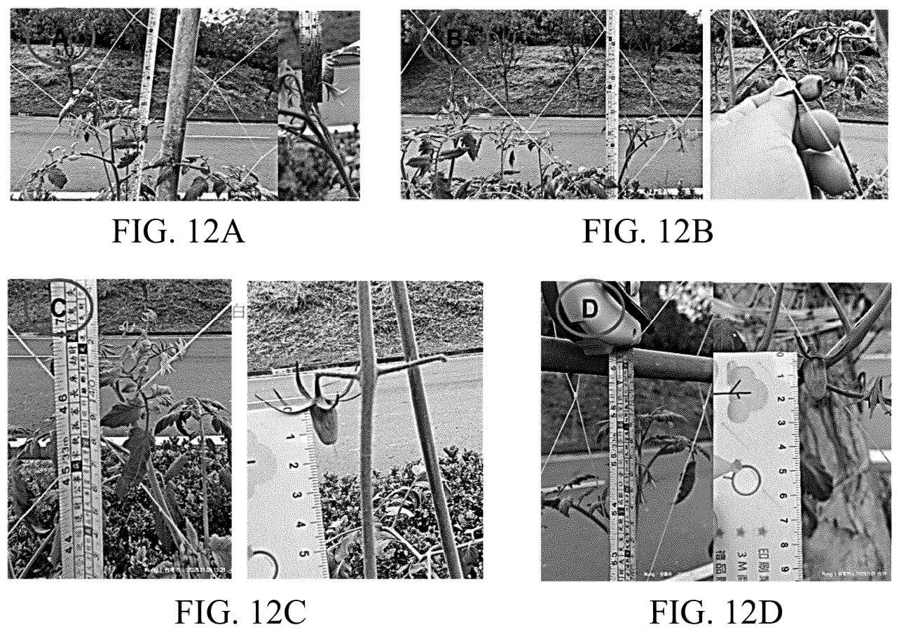

Further, observations were made on the outdoor growth conditions of the pots. FIGS. 12A to 12D show the growth results of small-fruited tomatoes in outdoor pots on the 21st day after planting. As shown in FIG. 12A, the potted plants of the control group did not have any flower buds. As shown in FIG. 12B, the potted plants of the Comparative Example group 2 had two flower buds, a plant height of 136 cm, and the largest fruits, but exhibited calcium deficiency. As shown in FIG. 12C, the potted plants of the Comparative Example group 1 had two flower buds, a plant height of 143 cm, with comparable fruit number and size, and no calcium deficiency. As shown in FIG. 12D, the potted plants of the Example group had four flower buds, a plant height of 170 cm, significantly taller than the other three groups, and comparable fruit number and size, with no calcium deficiency.

To summarize the above, in accordance with the embodiments of the present disclosure, a method of preparing an adjustable organic fertilizer is provided. The method can adjust the feed composition of organic waste according to the growth needs of the target crops (for example, the required ratio of nitrogen, phosphorus, and potassium) to form a composite feeding substrate with appropriate fertilizer content, thereby optimizing the anaerobic fermentation reaction, reducing high concentrations of ammonia nitrogen from inhibiting the metabolic pathways of anaerobic organisms. Accordingly, the decomposition and stabilization of organic matter can be accelerated and converted into organic fertilizer with a fertilizer composition suitable for target crops. In particular, the fertilizer composition of the obtained organic fertilizer is adjustable by adjusting the composite feeding substrate. That is, the fertilizer composition of the organic fertilizer can be optimized according to different target crops. Therefore, the organic fertilizer formed by the method provided by the embodiments of the present disclosure does not need to undergo aerobic composting and adding animal and plant meal to adjust the fertilizer content, and can be directly applied to the soil for irrigation.

Although some embodiments of the present disclosure and their advantages have been described as above, it should be understood that various changes, substitutions and alterations can be made herein without departing from the spirit and scope of the disclosure as defined by the appended claims. In addition, each claim constitutes an individual embodiment, and the claimed scope of the present disclosure also includes the combinations of the claims and embodiments. The scope of protection of the present disclosure is subject to the definition of the scope of the appended claims.

What is claimed is:

1. A method of preparing an adjustable organic fertilizer, comprising the following steps:
providing a ratio of nitrogen, phosphorus, and potassium required for a target crop and collecting a plurality of wastes;
categorizing fertilizer concentrations of the same type of feeding substrates within the plurality of wastes separately, and adjusting a composition of the categorized wastes to form a composite feeding substrate according to the ratio of nitrogen, phosphorus, and potassium required for the target crop;
subjecting the composite feeding substrate to an anaerobic fermentation process, comprising:
introducing the composite feeding substrate into an acidification tank to obtain a first mixture, wherein the acidification tank comprises acidifying bacteria, a pH value of the acidification tank is between 3 and 7.5, and a temperature of the acidification tank is between 30° C. and 60° C.; and
introducing the first mixture into a methanogenesis tank to obtain a second mixture, wherein the methanogenesis tank comprises methanogen, a pH value of the methanogenesis tank is between 6 and 8, and a temperature of the methanogenesis tank is between 30° C. and 60° C.; and
separating the second mixture to obtain a liquid digestate and a solid digestate, wherein the solid digestate serves as an organic fertilizer.

2. The method of preparing an adjustable organic fertilizer as claimed in claim 1, wherein a carbon/nitrogen (C/N) ratio of the organic fertilizer is between 10 and 20.

3. The method of preparing an adjustable organic fertilizer as claimed in claim 1, wherein the method does not comprise an aerobic fermentation step.

4. The method of preparing an adjustable organic fertilizer as claimed in claim 1, wherein the solid digestate obtained from separating the second mixture serves as the organic fertilizer without further adjustment for the fertilizer content.

5. The method of preparing an adjustable organic fertilizer as claimed in claim 1, wherein the target crop comprises vegetables, fruit trees/fruits, grains, legumes, root crops, plant raw materials, melons, prairie plants, lawns, spice plants, flowers, or a combination thereof.

6. The method of preparing an adjustable organic fertilizer as claimed in claim 1, wherein the plurality of wastes comprise livestock waste, agricultural waste, food waste, or a combination thereof.

7. The method of preparing an adjustable organic fertilizer as claimed in claim 1, wherein the acidifying bacteria comprises Defluviitoga, Thermoanaerobacterales, Syntrophomonadaceae, Synergistaceae, Limnochordales, Tepidimicrobium, Symbiobacterium, Syntrophaceticus, or a combination thereof.

8. The method of preparing an adjustable organic fertilizer as claimed in claim 1, wherein the methanogen comprises Methanoculleus, Methanospirillum, Methanothermobacter, Methanosarcina, or a combination thereof.

9. The method of preparing an adjustable organic fertilizer as claimed in claim 1, wherein the step of categorizing fertilizer concentrations of the same type of feeding substrates within the plurality of wastes separately comprises:
analyzing total nitrogen (N), total phosphoric anhydride (P₂O₅), and total potassium oxide (K₂O) of the plurality of wastes.

10. The method of preparing an adjustable organic fertilizer as claimed in claim 9, wherein the analysis of the total nitrogen, total phosphoric anhydride, and total potassium oxide of the plurality of wastes is conducted using an inductively coupled plasma optical emission spectroscopy (ICP-OES).

11. The method of preparing an adjustable organic fertilizer as claimed in claim 1, wherein the step of adjusting a composition of the categorized wastes to form a composite feeding substrate according to the ratio of nitrogen, phosphorus, and potassium required for the target crop comprises:
adjusting the feeding substrate amount of the plurality of wastes through mass balance calculations so that the concentrations of nitrogen, phosphorus, and potassium in the composite feeding substrate are higher than the concentrations required by the target crop.

12. The method of preparing an adjustable organic fertilizer as claimed in claim 1, wherein the fertilizer content of the organic fertilizer is substantially less than the fertilizer content of the plurality of wastes.

13. The method of preparing an adjustable organic fertilizer as claimed in claim 1, wherein a concentration ratio of nitrogen to phosphorus to potassium in the composite feeding substrate is in a range of 1 to 6:1 to 6:1 to 6.

14. The method of preparing an adjustable organic fertilizer as claimed in claim 1, wherein a total solid content (TS) of the composite feeding substrate is between 5% and 15%.

15. The method of preparing an adjustable organic fertilizer as claimed in claim 1, wherein the ammonia nitrogen concentration of the composite feeding substrate is between 800 mg/L and 4000 mg/L.
